(19) United States
(12) Patent Application Publication
(10) Pub. No.: US 2005/0150504 A1
(43) Pub. Date: Jul. 14, 2005

(54) Method and device for addressing sleep apnea and related breathing disorders

(76) Inventors: David W. Heeke, East Lansing, MI (US); Walter Britt, Wayland, MI (US)

Correspondence Address: DENISE M GLASSMEYER;YOUNG & BASILE, P.C., 3001 W. BIG BEAVER RD., SUITE 624, TROY, MI, 48084-2813, US

(21) Appl. No.: 10/757,923

(22) Filed: Jan. 14, 2004

(51) Int. Cl. A61F 5/56

(52) U.S. Cl. 128/848

(57) ABSTRACT

A device which is removably insertable in the mouth for facilitating breathing while sleeping which provides a clear unobstructed airway by protrusive positioning of the mandible and/or delivery of pressurized air to the back of the mouth. The device has upper and lower tooth-contacting members and an airway defined between them.

### CROSS REFERENCE TO RELATED APPLICATION AND FIELD OF THE INVENTION

This application is a divisional of U.S. Ser. No. 10/172,315 filed Jun. 14, 2002, which claims the benefit of U.S. Provisional Application Ser. No. 60/298,997, filed Jun. 18, 2001. The invention relates to oral appliances and methods for making same. More particularly the present invention relates to oral appliances which can facilitate breathing while sleeping.

### DESCRIPTION OF THE RELEVANT ART

Difficulty breathing while sleeping often manifests itself as snoring or, the more serious condition, obstructive sleep apnea. Snoring is a condition affecting approximately 40% of the adult population, while obstructive sleep apnea affects approximately 7% of the adult population. Although snoring can occur as a result of a physical anomaly, such as enlarged tonsils or adenoids, generally, snoring occurs during sleep because the muscles of the upper throat relax. As a person breathes, the turbulence of the air causes a flutter valve effect on the soft tissues of the upper throat. The vibration resulting from the flutter valve effect of the soft tissues of the upper throat causes snoring sounds.

Airway occlusion during sleep may cause cessation of breathing (apnea) and can lead to undesirable physiological symptoms. Sleep apnea is due to the obstruction of the upper airway which produces short episodes of breathing stoppage that characterizes apnea. Frequent arousals during the night occur when the user awakens in order to overcome the airway blockage. As a result, sleep apnea can contribute to excessive daytime sleepiness as well as high blood pressure, strokes or cardiac arrest.

Airway occlusion may be caused by decline in upper airway dilator muscle tone, particularly in the genioglossus muscle. Other conditions such as excess pharyngeal tissue and edema may act as contributing factors. Certain sleep positions can exacerbate the condition. For instance, sleeping on the back can add to the apnea problem due to the added effect of gravity on the tongue. In many instances, sleep apnea is most pronounced during the inspiratory phase of breathing.

Although such severe steps can be taken to alleviate snoring or the more serious condition of sleep apnea as performing a surgery, such as a tracheotomy, to ensure adequate air exchange; it is desirable to provide a treatment that is non-surgical, non-evasive, comfortable, and not unsightly. It is also advantageous to provide a treatment strategy which provides the user with prior knowledge of its effectiveness. Such prior knowledge further ensures compliance.

Currently, there are a number of therapeutic devices which can be used which do not require surgery. U.S. Pat. No. 5,562,106 to Heeke et al discloses a device for releasable insertion into a user's mouth to position the mandible in a protrusive open orientation thereby providing a clear unobstructed airway and eliminating or substantially alleviating snoring. The device disclosed in Heeke et al is adapted to fit over the upper four posterior teeth on both sides of the jaw and the corresponding lower posterior teeth in a deformable, flexible retentive manner. When in position, the device provides protrusive jaw movement sufficient to open the airway and eliminate snoring sounds. Additionally, the device provides a vertical opening between the upper and lower jaw sufficient to provide an adequate airway opening.

U.S. Pat. No. 4,715,368 and Reissue No. 33,442 issued to George discloses an oral device to prevent the closing of the breathing passage. The oral device consists of a one-piece mouthpiece having a front beak housing with an orifice airway therein. The oral device is custom-fitted and anchored to appropriate molars with wire clasps and a guide. Flanges are used to depress and constrain the tongue of the user to prevent closure of the breathing passage. This constraint on the tongue can prove uncomfortable to certain users and discourage use of the device.

U.S. Pat. No. 5,092,346 issued to Hayes and Meade discloses a dental device which grips all upper teeth forward of the pre-molars and has a downwardly extending ramp against which the lower teeth engage during sleep. An aperture in the device between the upper portion and the lower portion facilitates the passage of air for mouth breathing and orients the tongue forward in the mouth.

U.S. Pat. No. 5,003,994 issued to Cook discloses an oral device to forcibly position the mandible forward. The device has a rigid shell with an appliance socket structure adapted to engage the upright portion of tooth and gum of either the top or bottom jaw. The device also includes a cam structure which extends downwardly and has a pliant tooth contacting material such as silicone. Similar to the Hayes and Meade device, this oral device includes a central breathing aperture.

In cases of sleep apnea, it is often necessary to provide a steady stream of continuous air to maintain an unobstructed air pathway. The aforementioned devices do not make provision for the delivery of continuous positive air pressure. Typically such continuous positive air pressure (CPAP) devices include means for delivering a steady stream of pressurized air to the airway of the user. Such devices may include devices which are positioned exteriorly on the face and attach to the nose of the user or affix around the face to cover the nose and/or mouth of the user. Exteriorly positioned devices do not compensate for redundant tissue and laxity of dilator muscle tone.

U.S. Pat. Nos. 5,950,624 and 5,884,625 both to Hart are directed to devices which are removably positioned in the oral cavity of the user to deliver positive air specifically directed at the user's retroglossal area. The device includes a hollow elongate body adapted to be received in the oral cavity of the user and a lip extending from the elongate cavity to be grasped between the user's teeth in a manner which orients the user's teeth slightly apart and the user's lower jaw slightly forward from the relaxed jaw position. The device is coupled to a tubular member which is connected to an air pressure source.

Oral CPAP appliances which can be removably positioned in the oral cavity and could enhance user comfort and insure precision fit would be highly desired. Enhanced user comfort would and precision fit would encourage compliance and proper use. Additionally, as the device is to be worn for extended hours during sleep, it is also desirable that the device be one which would protect teeth and associated tissue from deleterious effects due to drying and the like. Finally, it would be desirable that the device have the potential to provide some limited alleviation of the symptoms of sleep apnea in certain users for limited time periods even if used without connection to a source of continuous positive air pressure.

### SUMMARY OF THE INVENTION

The present invention is a device for removable insertion into a user's mouth to provide a clear unobstructed airway to facilitate breathing while sleeping. This can be accomplished by the delivery of pressurized air and/or positioning the mandible in a protrusive open orientation. The device is composed of polymeric material suitable for use in human oral cavities which is flexible, lightweight and adapts to the contours of at least one or more teeth to permit a self-retentive frictional contact between the device and the associated teeth. The device includes a central body which is adapted to be removably inserted into the oral cavity of the user. The central body includes a hollow central conduit for conveying pressurized air from an external pressurized air source to the back of the user's throat. The device also includes an opening located proximate to the lingual area of the user's mouth and distal to the device opening proximate to the back of the user's throat.

When in position, the device preferably provides protrusive jaw movement sufficient to assist in opening the airway. Additionally, the device provides a vertical opening between the upper and lower jaw sufficient to permit an adequate airway.

The device may be of unitary construction or may comprise separable first and second members. In the embodiment comprising first and second members, the first member has a teeth-engaging region adapted to be removably positionable over the teeth of the wearer and a central palate element extending between the various portions of the teeth-engaging region to be positioned in overlying relationship proximate to the hard palate of the wearer. The second member of the two-member embodiment includes a central element adapted to be positioned below the palate element of the first member where the first and second members are in connected engagement to one another. When in such engagement, the palate element and the central element act to define the hollow central conduit for delivering pressurized air to the back of the user's throat. Means for releasably connecting the device to a source of external air are also integrally connected to the device, preferably to the lower member.

The device of the present may be configured to provide for measured protrusive adjustment of the lower jaw relative to the oral cavity through a series of precisely positioned attachment means located at measure intervals intermediate between upper and lower biting surfaces.

Other objects, advantages and applications of the present invention will become apparent to those skilled in the art when the following description of the best mode contemplated for practicing the invention is read in conjunction with the accompanying drawings.

### BRIEF DESCRIPTION OF THE DRAWINGS

The description herein makes reference to the accompanying drawings wherein like reference numerals refer to like parts throughout the several views, and wherein:

FIG. 1 is a partial elevation and cross-section of the human head and neck showing the oral device of the present invention in place;

FIG. 2 is a perspective exploded view of one embodiment of the oral device of the present invention;

FIG. 3 is a front view of the oral device of the present invention in position in the mouth;

FIG. 4 is an upper view of the oral device of the present invention;

FIG. 5 is a lower view of the oral device of the present invention;

FIG. 6 is a perspective view of an alternate embodiment of the present invention in which upper and lower tooth-engaging members are positionally adjustable relative to one another;

FIG. 7 is a detail drawing of a fastening member for securing upper and lower tooth engaging members;

FIG. 8 is an upper view of a fastening member of the device of FIG. 6;

FIG. 9A is a side cross-sectional view of a fastening track of the present invention;

FIG. 9B is a side cross-sectional view of an alternate fastening track of the present invention;

FIG. 9C is a side cross-sectional view of an alternate fastening mechanism of the present invention;

FIG. 10 is a detail drawing of an attachment pin suitable for use in the mechanism of FIG. 6;

FIG. 11A is an exploded perspective view of the mandible member of the alternate embodiment of FIG. 6;

FIG. 11B is a bottom view of the maxillary member of FIG. 6; and

FIG. 12 is a detail view of the alternate attachment mechanism.

### DESCRIPTION OF THE PREFERRED EMBODIMENT

The oral device for treating sleep apnea and related breathing disorders is generally indicated by numeral 10. The device 10 is composed of a polymeric material constructed from a suitable polymeric material or combination of materials which provides a finished oral device which is flexible, light-weight, durable and capable of adapting to the contours of the wearer's teeth to permit self-retentive frictional contact between the device and the associated upper or lower teeth. The polymeric material is, preferably a material approved by the U.S. Food and Drug Administration for use in the oral cavity. Preferably, the oral device of the present invention is composed of a polymethylmethacrylate resin independent of or in combination with a methylmethacrylate resin and may also contain amine initiators, ethylene glycol and specific methacryloyloxyethanes. The polymeric material is flexible, lightweight, translucent and adapts to the contours of the teeth to permit a self-retentive frictional contact between the device and the associated upper and lower posterior teeth. The flexible material is trimmable to provide reduced retention where necessary and for more comfort; and will provide better usage by the user over the hard plastic material of prior devices. The material is durable and easily maintained, and can be cleaned by brushing and/or soaking; by way of example, suitable polymethylmethacrylate resin powders and methyl methacrylate base resin liquids from Ivoclar North America of Amherst, N.Y. Specific characteristics of these materials are enumerated in Table I and Table II.

TABLE I
Characteristics of Polymethylmethacrylate Resin Powders

| Contents | % Range |
|---|---|
| Polymethylmethacrylate | 60-100 |
| Benzoyl Peroxide | 0.5-1.5 |
| Titanium Dioxide | 0.1-1.0 |
| Characteristics: | fine clear pink dust-like particles with no odor |

TABLE II
Characteristics of Methyl Methacrylate Resin Liquids

| Contents | % Range |
|---|---|
| Methyl Methacrylate | 60-100 |
| Amine Initiator | 0.1-1.0 |
| 1,2 Bis (Methacryloyloxy) Ethane | 1-5 |
| Ethylene Glycol | 1-5 |
| Boiling Point: | 100° C. |
| Vapor Pressure (mm Hg): | 29 kPa |
| Vapor Density (air = 1): | 3.46 |
| Specific Gravity (H₂0 = 1 glcm³): | 0.95 |
| Flash Point (° C.): | 11.5 Closed Cup |
| Appearance and Odor: | clear colorless liquid, sharp odor |

The device 10 of the present invention may be a unitary device, i.e. molded in one piece, or, more preferably, may be composed of two separable pieces to increase flexibility of use and to permit proper and effective cleaning of the device and associated apparatus. In the preferred embodiment, the device 10 of the present invention comprises an upper member 12 also referred to as a palatal shell adapted to be secured to the region of the user's mouth at or near the rear teeth. The device also includes a lower or central plate member 14 which is removably connected to the upper member 12 or palatal shell and cooperatively acts with the palatal shell 12 to secure the device 10 in the mouth of the user in a removable manner. The device 10 of the present invention also includes means 14 for connecting the device to a source of pressurized air (not shown) and defines a central conduit C for delivering the pressurized air through the oral cavity to the rear of the mouth proximate to the throat.

In the preferred embodiment of the present invention, the palatal shell 12 has upper depressions and lower depressions adapted to releasably receive the upper teeth as well as lower posterior teeth, preferably including molars 20 and second bicuspid 22 of both the upper and lower jaw are retentively secured with forward teeth being engaged. The depressions provide biting surfaces 24 for the aforementioned teeth. A retentive portion 26 of the upper member 12 fits over the upper four posterior teeth on both sides of the jaw and the corresponding lower posterior teeth. The retentive portion 26 extends upwardly toward the gum line 28 of each respective tooth to provide a more secure fit and contact between the device 10 and the teeth.

A plate-like bridge 30 conforming to the back of the maxillary anterior teeth and the upper palate of the wearer joins the two respective upper biting surfaces 24. In the preferred embodiment, the plate-like bridge 30 extends from a rear terminus 34 proximate to transition between hard and soft palate in the user's mouth to a front edge 35 overlying the interior surface of the frontal upper incisors 38.

The plate-like bridge 30 has an upper face 31 and an opposed lower face 33. The plate-like bridge generally has a convex surface when viewed from the top. The convex surface typically has a geometry which will comfortably conform to the contours of the palate of the user. The lower face 33 has a correspondingly curved surface. The plate-like bridge 30 preferably has a thickness sufficient to provide flexible strength to the device of the present invention.

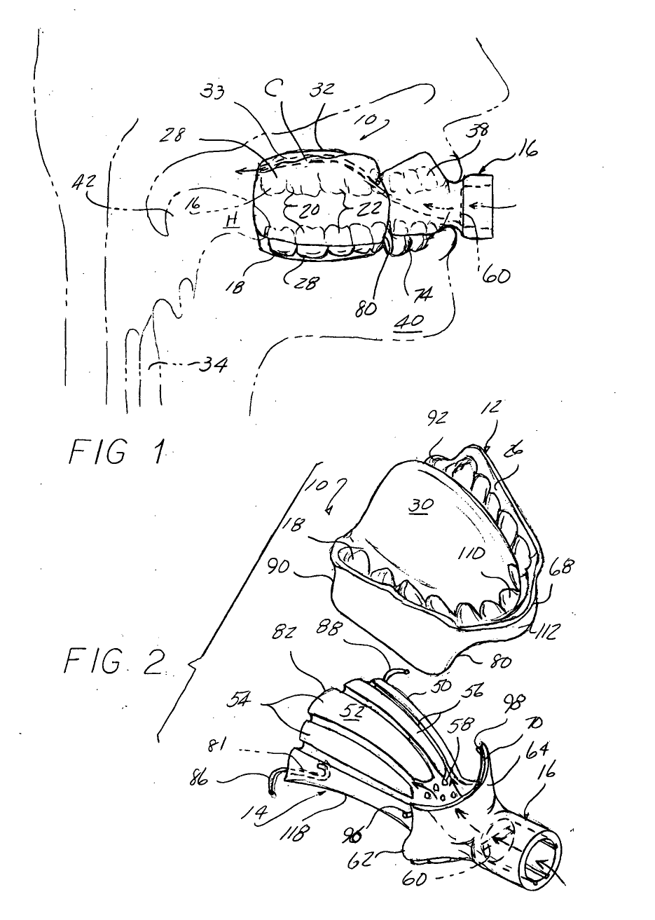

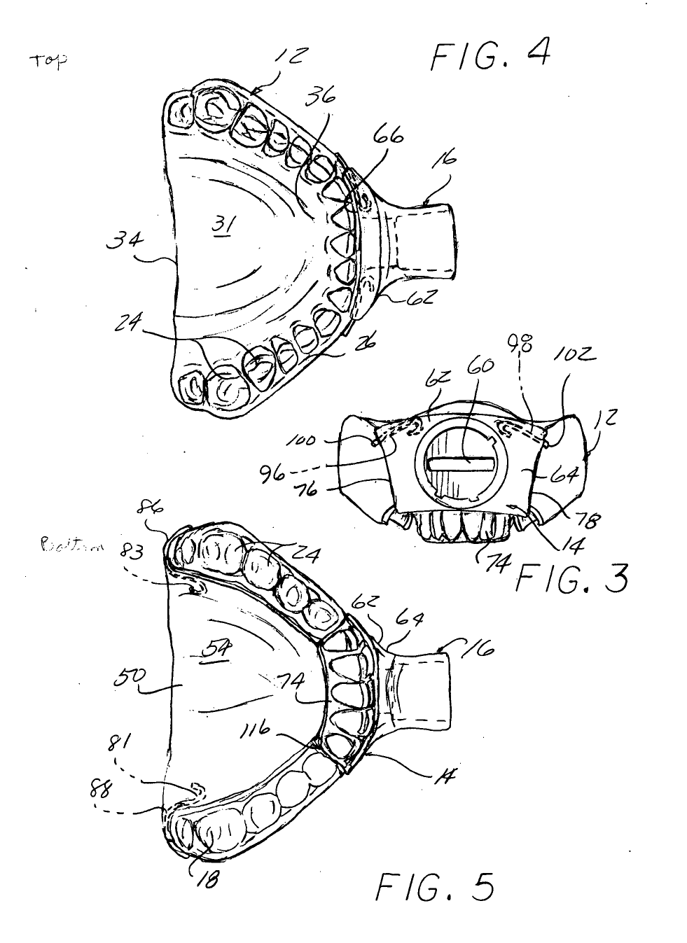

With the preferred embodiment, the biting surfaces are spaced at a height (H) and protrusive jaw 40 position sufficient to cooperatively maintain the airway opening. This provides an enhanced aperture in the mouth though which the continuous stream of pressurized air can be introduced. For the user who has an edentulous mouth, the oral device 10 can be molded to the gums where the back molars and second bicuspid would have been located. It is preferred that the device 10 encompasses the area of at least four back teeth, upper and lower, on each side of the mouth to provide sufficient amount of material included in the biting surface to provide adequate retentive area and to prevent a user with the nervous disorder bruxism from grinding on the oral device 10. As seen in FIGS. 1 and 2, the retentive portion 26 of the oral device 10 extends toward the gum line 28 of each respective tooth over the super bulge of each contacted tooth to provide a more secure fit and contact between the device and the teeth.

Once the oral device 10 is in place, the jaw 40 will be protruded outward. This naturally forces the tongue 42 forward, thereby providing an open passageway leading to the trachea 44 and esophagus (not shown). The oral device 10 of the current invention does not restrain and cause discomfort to the tongue.

When used to deliver pressurized air, the oral device 10 of the present invention also includes a lower or central plate member 14 which matingly corresponds to the upper member 12 to define a central air passage C through the device 10 from the region proximate to the frontal upper incisors 38 to the rear of the device proximate to the region where the soft palate and hard palate meet.

In the preferred embodiment, the device 10 of the present invention is adapted to conform to the contours and geometry of the individual user to the extent necessary to ensure effective retention of the device in the oral cavity of the user as well as the comforts and convenience of the user. Thus it is envisioned that at least portions of the device can be crafted from individual impressions to provide maximum conformity to the mouth of the individual wearer either by a process of dental impressions and casting or by any suitable method which will provide such conformity. While both upper and lower members may be individualized, it is contemplated that in many instances, the upper member will be individually contoured while to lower member may be available in more standard sizing.

The lower member 14 has a central plate 50 having an upper or inwardly facing surface 52 and a lower or outwardly facing surface 54. The central plate member 14, preferably, has a general contour which conforms significantly to the contour of the user's own palate to increase comfort and wearing ease. Preferably, the general contour of the central plate 52 will generally correspond to the contour of the plate-like bridge 30 of the associated upper member 12. However, the general contour of the central plate 50 may be any configuration which will facilitate air flow through the device 10 of the present invention.

Similarly, the geometry of the upper face 52 of central plate 50 typically is one which will facilitate the direction and channeling of continuous positive pressure air flow into the throat of the user at a location which maintains the open airway.

The upper face 54 may have at least one upwardly extending support projecting therefrom toward a terminus proximate or in contact with the lower face of the plate like bridge 30. At least one support is preferably contiguously joined to the central plate and are preferably formed or molded out of suitable polymeric material. At least one support is positioned to provide structural reinforcement to the central plate 50 to prevent collapse of central plate 50 against the lower face of the plate-like bridge during swallowing and the like.

At least one support may be suitable, configured to prevent collapse while maintaining adequate pathways for pressurized air flow through the channel define by upper member 12 and lower member 14. As shown in FIG. 2, the at least one support includes a plurality of ridges 54 which extend longitudinally from front to rear of the lower member 14 in the general direction of the flow of pressurized air as depicted by the flow arrows in FIGS. 1 and 2. The elongated ridges 54 may be of any suitable dimension which will prevent collapse during use; particularly during swallowing. The elongated ridges 54 are suitably configured to permit suitable air flow. Typically, the ridges 54 extend upward from the lower member 14 and terminate at a point below the proximate surface of the upper member 12 so as to permit a large, essentially unobstructed air channel for maximum air flow capacity through the device 10. As shown in FIG. 5, the elongated ridges 54 also define a plurality of subchannels 56 which can also serve to direct the flow of pressurized air from a region proximate to the front of the oral cavity to the terminal outlet of the device.

Thus, even during compression during swallowing episodes, air flow capability is maintained. The device 10 may also include at least one flow directing protrusion 58 extending upward from the upper face of the lower member 14 and located in the region proximate to air entry orifice 60. The at least one flow direction protrusion 58 extends upward from the upper face of the lower member 14 to a point at least proximate to the lower face of the associated upper member 12. The at least one protrusion 58 will be positioned in a manner which permits the direction of pressurized air in an efficient and effective manner through the device 10. Additionally, the at least one lower member 14 may also serve to prevent collapse of lower member 14 against upper member 12 in the manner previously described.

While the preferred embodiment of the device 10 of the present invention contemplates protrusions 58 and ridges 54 extending upward from the upper face of the lower member 14, it is also considered within the purview of the present invention for ridges, protrusions and other reinforcement and/or air directing elements 10 be integrally connected to the lower face of the upper member 12.

The lower member 14 also includes a contoured shield contiguously connected to and extending upward from the frontal region 64 of the central plate 50. The contoured shield 62 is a curvilinear member oriented in an essentially perpendicular position relative to the central plate 50. The shield member 62 has an outwardly oriented surface 64 from which the external air connection means 16 extends. Preferably the outwardly oriented surface 64 has a smooth contoured surface adapted to comfortably contact the inner surface of the user's upper lip.

The contoured shield 62 has an inner face 66 opposed to the frontal region 64. The inner face 66 is adapted to mutually receive the forward most region 68 of the upper member 12 and extend into mating contact or proximate relationship with the outer surface of the upper incisors of the user. The outer face 67 is contoured to fit in comfortable contact with the inner surface of the upper lip region to promote delivery of a continuous stream of pressurized air through the device 10 in an effective and efficient manner.

The contoured shield 62 terminates in an upper edge 70 which extends upward from the connection means 16 to a point which preferably covers the front upper incisor teeth. The contoured shield 62 also has an opposed lower edge region 72 adapted to extend in front of the lower incisor teeth of the user.

The lower edge 72 region is configured with an interior tooth contacting bite region 74 which preferably is contoured to receive and contain and cover the inner surface of the lower incisors where the device 10 is in position in the oral cavity of the user. In this manner, the labial and lingual surfaces of all teeth and associated gum regions are suitably isolated from the continuous stream of pressurized air. Such isolation protects teeth and associated gum regions from deleterious effects which may be associated with drying and the like.

The contoured shield 62 also has opposed side edges 76, 78 which are adapted to mutually contact associated surfaces of the associated upper member 12. Preferably the opposed side edges 76, 78 are configured to terminate at a line which extends to overlie the upper and lower incisors and cuspids. The lower tooth contacting bite region 74 is adapted to abut the frontal edges 80 of the respective bite regions 18 of the upper member 12.

The device 10 also includes suitable means for releasably attaching lower member 14 to upper member 12. Attachment means may be any suitable arrangement of channels, grooves and/or fastening member to anchor the two respective members 12, 14. In the preferred embodiment, the central plate 50 of lower member 14 has a terminal edge 82 adapted to be received in the interior of the user's mouth. Proximate to the terminal edge 82 are first means 84 for releasably anchoring the lower member 14 relative to the upper member. In the preferred embodiment, the first anchoring means 84 comprises a pair of flexible wire clips 86, 88 which contact and engage the rearwardly oriented surface 90, 92 of the upper member 10 rearward of the molars region. In the preferred embodiment, the flexible wire clips act cooperatively with the retentive portion of the upper member effectively anchor lower member of the device 10 into position. Flexible clips 86, 88 may have any configuration suitable for retention. As depicted in the drawings Figure, wire clips 86, 88 have an interior region 81, 83 embedded in the lower member proximate to the rearward edge 82 and a curvilinear central region 85, 87 and each terminates in an end region 89, 91 which is parallel to the interior region 81, 83 such that the rounded end of upper member 12 is received within the U region so defined such that the upper member 12 is retentively held relative to the respective clip.

The lower member of device 10 of the present invention also includes second means 94 for releasably anchoring the lower member of device 10 relative to the upper member 12 located proximate to the mouth opening and the front incisors in order to secure the front shield relative to the upper member. In the embodiment depicted in the drawing Figures, a pair of pins 96, 98 are embedded in front shield proximate to upper edge 70. In the preferred embodiment, pins 96, 98 protrude laterally outward from side edges 76, 78 and are adapted to be releasably received in mating bores 100, 102 located in upper member 12.

In the preferred embodiment, the frontal shield has an upwardly extending region which conforms to and contacts the front upper incisors of the user. In the preferred embodiment of the device 10 of the present invention, the upwardly extending region of the frontal shield acts cooperatively with the front edge 68 of the upper member to releasably contact and encase the incisors and any proximate teeth as necessary and thereby provide secure anchorage for the front section of the device 10 of the present invention located proximate to the mouth opening. In the preferred embodiment, the front edge 68 of the upper member 12 is adapted to completely cover the gum region proximate to the incisors and other associated teeth in the region such as the cuspids or eyeteeth. The front edge region 68 is in contact with the associated when assembled, the front edge region 68 is in contact with the associated region of the lower member 14. Preferably, the front edge region 68 is configured with a biting surface 110 which will cover the biting edge of all associated front teeth.

When the upper member 12 and lower member 14 are in secure connected relationship. The forward portion 112 of front edge region 68 is urged into abutting relationship with the inner surface of frontal shield 64 in a manner and at a position such that the region of the lower face 33 plate-like bridge 30 proximate to the front edge region 68 is positioned about pressurized air inlet 60 and in sealed abutting relationship to the inner surface of frontal shield 64. Thus the air flow channel C is defined by the lower surface 33 of upper member 12 and the upper surface 52 of lower member 14.

When upper member 12 and lower member 14 are in secure contactable relationship, the front edge 112 of the upper member 12 and the associated region of the lower member 14 form an essentially air tight connection there between. In this manner, the teeth and sensitive gum region are further protected from deleterious effects associated with the introduced pressurized air such as drying or the like. Additionally the air tight junction prevents seepage or air leaks which would compromise the effectiveness and efficiency of the pressurized air delivery to the throat region of the user.

In the preferred embodiment, lower member 14 of the device 10 of the present invention has a biting surface located anterior to the channel 116 and junction point between the upper member 12 and the lower member 14. The biting surface 58 is of sufficient size to provide that a sufficient amount of material be included in the biting surface to provide adequate retentive area and to prevent a user prone to teeth clenching from prematurely grinding on or degrading the oral device 10.

To effectively matingly join the upper member 12 and the lower member 14, it is preferred that the device 10 of the present invention include a junction integrally formed in the body of the upper member which permits the separable mating of the two respective members in a manner which defines a suitable central air conduit C though which introduced pressurized air may be conveyed. To accomplish such mating, the upper member 12, preferably, includes a groove or channel 116 located proximate to a junction between upper plate 30 and respective retentive regions and adapted to receive side edges 118 of the lower member 14. Groove 116 is configured to receive respective side edge 118 in an essentially air tight manner. Typically the engagement between the two respective elements would be a snap fit or telescopic arrangement.

The groove 116 of the lower member 112 preferably extends onward to the anterior surface proximate to the back molar region of the upper member 12 of the device to matingly receive the flexible wire clips 86, 88 projecting from the anterior of the lower member 14. Engagement of the flexible clips 86,88 into the respective anterior regions of grooves 116 further secures the contact between the two respective members and prevents lateral slippage of the devices relative to one another.

Assembly of the device 10 is preferably accomplished prior to insertion into the mouth of the user. Due to the inherent flexibility of the material of construction of the two elements, the upper member 12 can be flexed to accommodate positioning of the lower member 14 into the grooves defined in the shell. Pins 96, 98 are inserted in respective bores 100, 102 located in upper member 12 while clips 86, 88 are urged into engagement with associated anterior regions of the upper member 12. When disassembly is required, the process can be reversed. Once assembled, the device 10 can be positioned in the mouth of the user by snap fit over the associated molars and bicuspids. Generally, the device is initially positioned on the associated upper back teeth. The device is, then brought into engagement with the frontal incisors and cuspids. Finally, the lower jaw is oriented to engage the associated lower cuspids and molars into defined positions in the palatal shell thereby positioning the mandible in position. In the preferred embodiment, the mandible is brought into a protrusive position which further facilitates opening of the airway. However, it is also considered to be within the purview of the present invention that the device be configured so to maintain the jaw in a non-protrusive position if it is determined that protrusion is not required to address and abate symptoms of sleep apnea.

Additionally it is envisioned that, in certain instances, under appropriate supervision, the upper member 12 may be employed for limited periods such as when traveling or in users whose sleep apnea varies in severity due to seasonal allergies and the like. In situations where the user is experiencing unobstructed breathing, it may be feasible for a user to wear the upper member 12 alone and experience sufficient symptomatic relief. However it is to be understood that the ability to use upper member 12 apart from the lower member 14 is, by no means, to be construed as endorsement of such use for the treatment of sleep apnea without the instruction and prior approval of medical professionals familiar with the situation of the particular user.

The device 10 of the present invention also includes means 16 for connecting the device 10 to a source of pressurized air (not shown). Preferably, the connection means 16 is integrally connected to lower the lower member 14 as shown in the drawing Figures. However, it is within the purview of the present invention that the connection means 16 may be connected to the upper member 12 if desired or required.

As depicted in the drawing Figures, the connection means 16 projects outward beyond the lips of the user to define a hollow channel 17 through which air may pass. The channel 17 has an outer region 19 adapted to releasably receive a suitable hose or other mechanism for conveying pressurized air to the device 10 of the present invention (not shown).

The connection means 16 may project outward beyond the lips at any angle or orientation which will facilitate connection to the source of pressurized air and conveyance of pressurized air through the connection means with the maximum possible user comfort. Size and shape can also be varied depending upon the best approach for receiving air from the pressurized air source.

As depicted the connection means 16 includes a sleeve 120 which is contiguously joined and extends outward from the front face 64 of shield 62. The sleeve 120 may have any suitable cross-sectional configuration such as the cylinder depicted in the drawing Figure. The configuration of sleeve 120 will preferably be on which will facilitate suitable air flow and provide an appropriate and comfortable connection between the source of pressurized air and the device 10.

As depicted the sleeve 120 is a hollow cylindrical member 122 having an outer air inlet 124 and a central hollow body 126 in fluid communication with the air opening 60. The interior geometry of the central hollow shaft 126 will be any configuration which will facilitate essentially non-restrictive air flow therethrough.

As depicted, the outer air inlet 124, is adapted to relescopingly receive a hose or other suitable air connecting conduit (not shown). Thus, the outer air inlet 124 may include suitable locking detents 218 to receive and position suitable have connectors relative thereto.

The two-piece device 10 of the present invention is, preferably, suitably configured such that the upper member 12 may be employed without the use of the lower member 14 in certain specific situations. When the upper member 12 is employed alone, it is preferred that the upper member to be configured to achieve appropriate mandibular protrusion. Examples of situations in which the upper member 12 could be worn alone include initial familiarization of the device 10 with the user. In such situations, the upper member 12 may be worn independently to determine and increase user comfort and to tolerance of the entire device 10.

The present invention is also directed to a device having means for variably adjusting a separate mandible contacting section relative to a maxillary contacting section. As depicted in FIG. 7, the alternate embodiment of the present invention comprises a device 210 having an upper maxillary contacting section 212 and a lower mandible contacting section 214. The device also includes means 216 for joining the upper maxillary contacting section 212 to the lower mandible contacting section 214. In the preferred embodiment, the joining means 216 has a capability for adjustably positioning the upper maxillary contacting member 212 relative to the lower mandible contacting member 214. In this manner, the mandible or lower jaw 218 can be placed in appropriate protrusive engagement relative to the upper jaw.

It is contemplated that adjustable device 210 can be used successfully with the lower member 14 described previously to provide a mechanism for delivering a steady stream of continuous pressurized air as desired or required. Thus, the device 210 of the present invention may include any and all features necessary to permit the engagement of lower member 14. It is also contemplated that the device 210 of the present invention can be employed independent of lower member 14 in certain situations. These situations can include an adaptation interval during which the user is gradually accommodated to the device 210 of the present invention. Additionally, it is contemplated that the device 210 of the present invention can, possibly, be employed by individuals who do not require administration of continuous pressurized air. Thus, the device 210 of the present invention may be employed as a mechanism for abating or eliminating snoring.

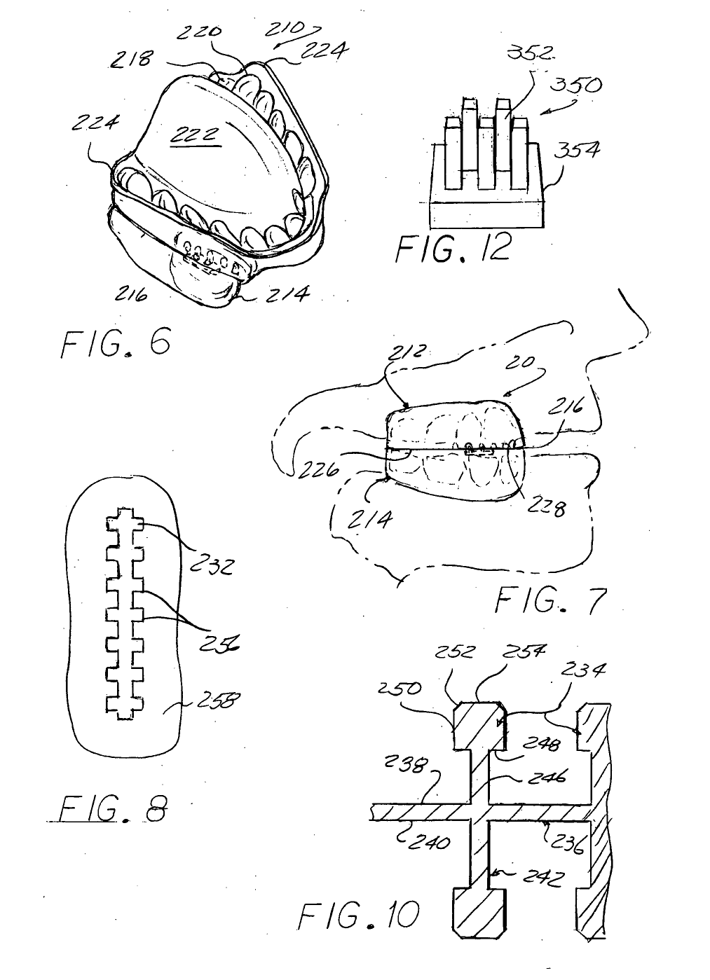

As depicted in FIGS. 6 and 7, the upper maxillary contacting portion 212 includes appropriate bite surfaces such as those illustrated at 218. The bite surfaces and upwardly extending outer members 220 are configured to provide flexible retentive positioning of the maxillary contacting portion 212 of the device 210 of the present invention to the associated teeth. Specifically, it is contemplated that the upper retentive portion 220 extend upward from the bite surface 218 to accommodate and encase the super bulge of at least two opposed rear teeth such as molars. The device 210 depicted in FIGS. 6 and 7, the flexible retentive portion 220 is adapted to extend over the four anterior upper teeth on both sides of the user's mouth.

The maxillary contacting portion 212 of device 210 as depicted in FIG. 6 also includes an upper palatal member 222. As shown in FIG. 6, the upper palatal member 222 is configured to be positioned in overlying relationship over significant portions of the hard palate of the user. A full palatal member 220 is preferred in situations where the device will be used to administer continuous air pressure. However, it is also contemplated that the size of the palatal member can be reduced in situations where the device 210 is used as a mechanism for eliminating or minimizing snoring.

The anterior bite regions 224 are configured to have an essentially flat lower face opposed to bite regions 218. The flat lower face 226 is adapted to be positioned in either proximate or contacting relationship to an upper surface 228 located on mandible contacting member 214. The lower surface 226 has a suitable junction or contacting means 230 adapted to positionally and releasably join the mandible contacting member 214 to the maxillary contacting member 212.

The mandible contacting member 214 and the maxillary contacting member 212 may be joined by any suitable connecting means. The connecting means of choice will, preferably, be one which permits adjustable connection of one member relative to another such that the degree of protrusion of the jaw member can be altered to account for user comfort and optimum airway clearance.

The connection means is preferably composed of retention pins or rods which are adapted to attach to one member of the device and extend to essentially vertically into the mating member. As depicted in FIG. 13, the connection means is a plastic plate member 350 having at least one geometric rib 352 extending contiguously from base 354. When employed, base 254 is adapted to be received in a suitably configured opening or orifice in either mandible contacting member 212 or maxillary contacting member 214. Base 254 may be attached by any suitable means to the associated member.

Each rib 252 can be either contiguous to associated ribs or separate there from. The ribs are adapted to be received in mating apertures in the opposed member 212 or 214. The ribs can be moved relative to apertures to obtain the desired protrusion. It is also contemplated that appropriate ribs can be removed during the adjustment stage to obtain protrusion as desired or required. The apertures may be reinforced by appropriate plastic sleeves as necessary.

The connection means may be composed of any suitable material such as various metals or polymers with essentially non-reactive materials such as Nylon being preferred.

A variation on connection means is presented in FIG. 12, where the connection means includes a plurality of spaced apertures 232 located in the upper face. Such apertures are adapted to receive an upwardly protruding members such as head 234 shown in FIG. 9C.

At the minimum, the attachment means 230 may be any mechanism which can suitably extend into an appropriate attachment receiving means defined in the lower face 226 of maxillary contacting member 212. While it is contemplated that this can be a single pin mechanism such as pin 234, it is desired that the attachment mechanism be one which will minimize or eliminate pivoting or rocking of the maxillary contacting member 212 relative to the mandible contacting member 214 as a result of swallowing, tooth grinding and the like. Thus, in the preferred embodiment, the attachment mechanism 230 preferably comprises at least two upwardly protruding pins positioned in spaced relationship to one another to minimize pivoting and rocking thereby.

The attachment mechanism 230 may be affixed to either mandible contacting member 214 or maxillary contacting member 212 in any suitable, permanent or semi-permanent manner. As depicted in FIG. 9C, attachment mechanism 230 is affixed to mandible contacting member 214 by a suitable adhesive or other member. However, to provide greater orientation options, connection member 230 can be affixed in a semi-permanent or movable means. In this more preferred variation, attachment member 230 includes a lower plate 236 having an upper face 238 and an opposed lower face 240. Faces 238 and 240 are adapted to be positionable and contact with faces 226 and 228 of maxillary contacting member 212 and mandible contacting member 214, respectively. A plurality of pins 234 are integrally connected to the plate 236 and extend upward from face 238 to a predetermined height sufficient to provide appropriate anchorage to the device relative to the upper mandible contacting member. The number of pins 234 which extend upward from face 238 and their orientation relative to one another is that sufficient to provide such anchorage. Thus, the number of upwardly extending pins 234 may be one or more as required to prevent rocking, pivoting or the like. As depicted in FIG. 11, two pins 234 extend upward base 238. However, it is within the purview of this invention to include additional upwardly extending pins as desired or required.

Each upwardly extending pin 234 is adapted to be received in an appropriate orifice 232 configured in the lower face 226 of mandible contacting member 212.

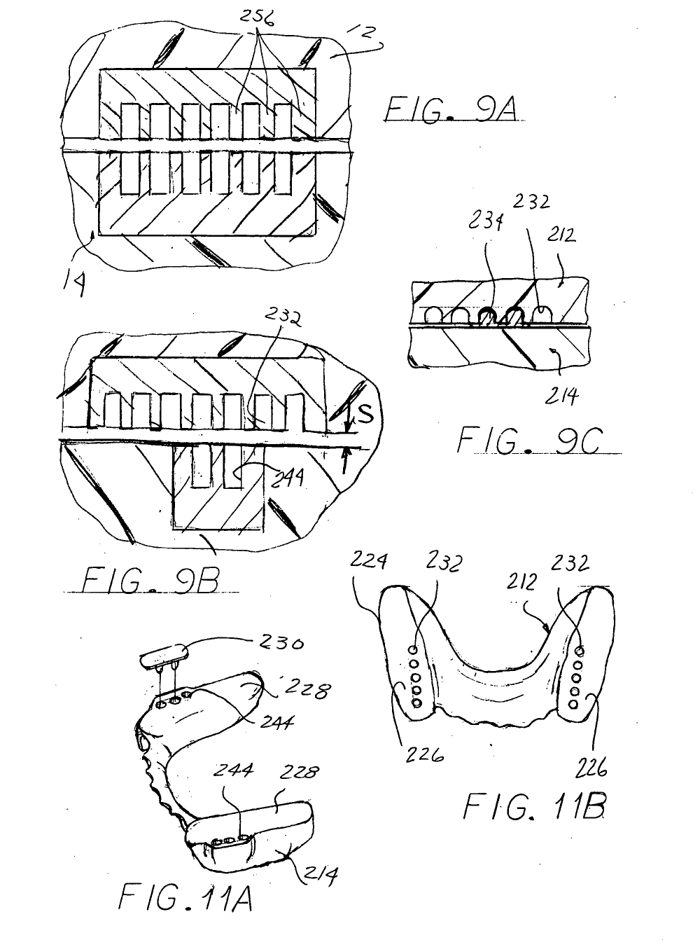

The connection member 230 as illustrated in FIG. 10, also includes at least one pin 242 which extends downward from face 240 to a predetermined position. Downwardly protruding pin 242 is adapted to be received within pin receiving orifices 244 located in the upper face 228 of lower mandible contacting member 214. The number of downwardly protruding pins 242 is that sufficient to prevent or minimize rocking or pivotal movement of the upper maxillary contacting member 212 relative to the lower mandible contacting member 214. Preferably, the downwardly protruding pins 242 will be of a number and an orientation sufficient to achieve this end. As depicted in FIG. 11, it is preferred that the connection member 230 include two downwardly protruding pins 242.

The pins 234 and 242 can be configured in any manner suitable to be received and anchored relative to the pin receiving orifices 232, 234 respectively. As depicted in FIG. 10, each pin receiving orifices 232, 234 respectively, has a central stem 246 having a first and contiguously joined and extending outward from the respective surface 238 or 240. In the preferred embodiment, the pin 246 is integrally formed with the base 236 to ensure optimum strength and support.

The end of pin 246 distal to the respective face 238, 240 is contiguously joined to an outer shoulder 248. The stem 246 can have any suitable cross-sectional geometry sufficient to permit insertion and secure engagement of the pin 234, 242 in its respective pin receiving orifice. In the preferred embodiment, the stem members each have a cylindrical geometry.

The shoulder 248 is, preferably, contiguously joined to the stem 246 in an essentially perpendicular manner. The shoulder layers to an outer diameter which is greater than the cross-sectional diameter of the stem 246. The shoulder forms the proximate surface of a head member 250 which has a cross-sectional diameter sufficient to the engagingly received in the respective pin receiving orifices. Preferably, the diameter of the head 250 is cylindrical in lateral cross-section and will have a diameter which is at least 20% greater than the diameter of the stem region 246, with diameters greater than approximately 50% being preferred.

The head region 250 preferably, has a frustoconical or oblique upper edge 252 and an essentially flat surface 254 which is oriented in an essentially parallel relationship to surface 238 or 240.

In this manner, as the pin 234 is inserted into the suitable orifice 232, frustoconical edge 252 facilitates such inward movement by triggering a slight deformation of the surrounding polymeric material. Once in position, the elastic memory of the polymeric material exerts itself and the pin is held in position by shoulder 248. The pin receiving orifices 232 and 244 can have any suitable configuration suitable for receiving the associated pin therein.

As depicted in FIGS. 11 and 12, the orifices 232 and 244 are a plurality of apertures positioned in essentially linear relationship on the respective surfaces 226 and 228. In the preferred embodiment, the upper maxillary contacting member 212 will have a series of engagement orifices which are located in the general proximity of the first bicuspid region and extend backward therefrom. The lower pin receiving orifices 244 are generally located in the region between the first molar and the first bicuspid in a manner which will facilitate orientation of the upper maxillary contacting region 212 relative to the lower mandible contacting region. Orientation of the two respective members which is so facilitated includes positioning which ranges from the normal bite of the user to a protrusive orientation of the mandible sufficient to provide an open airway in the user.

In the preferred embodiment, the number of lower pin receiving orifices 244 is sufficient to accommodate movable positioning of the contacting member 230 relative to the upper face 228. Typically, the number of positions accommodated is between 2 and 5. In the preferred embodiment, the number of pin receiving orifices 232 located in the lower face 226 of the upper member 212 is also sufficient to provide movable spaced positioning of the connecting member 230 relative to the maxillary contacting member 212. Preferably, the number of pin receiving orifices located in the upper member is sufficient to provide at least three different relative orientations, with a number sufficient to provide five or six being even more preferred.

The pin receiving orifices 232 and 244 can be individual orifices positioned in the respective members. Additionally, it is contemplated that the pin receiving orifices can be present as a series of individual interconnected ratchets 256 which are formed in the respective members. It is also considered within the purview of this invention that the pin receiving member having a plurality of individual ratchets 256 can be a separately machined and formed member which is adapted to be securely and permanently positioned in the body of the respective maxillary contacting member 212 or mandible contacting member 214. The individually inserted members 258 can either be of lengths similar to one another as depicted in FIG. 9A or may be configured such that one region, for instance the member positioned in the lower mandible contacting member is smaller and has fewer ratchets 256 than its respective member.

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

1. An oral device removable insertable in the mouth of a user for facilitating breathing while sleeping comprising:
an upper member, the upper member having upper and lower depressions and right and left sides corresponding to the location of at least back molars and second bicuspids configured to fit a mouth of a user, said upper and lower depressions forming right and left biting surfaces wherein said biting surfaces are spaced at a height to force the mouth open the upper member further having a plate-like bridge conforming to the back of the maxillary anterior and the upper palate of the mouth, said bridge joining the biting surfaces;
a lower member positioned in underlying spaced relationship relative to the plate-like bridge of the upper member, the lower member having a lateral member, the lateral member having an upper surface oriented toward the plate-like bridge, a lower surface opposed to the upper surface, and at least one curvilinear side surface extending from a frontal location to a terminal location proximate to the maxillary anterior of the user's mouth, the at least one side edge adapted to engage associated interiorly oriented regions of the upper member, the lower member positioned relative to the upper member so as to form an air passage for conveying introduced auxiliary pressured air from the mouth opening to the anterior of the mouth of the user.

2. The oral device of claim 1 wherein the lower member is in permanent contact with the upper member.

3. The oral device of claim 1 wherein the upper member has retentive portions extending over the super bulge of each of the back molars and second bicuspids.

4. The oral device of claim 1 wherein the flexible shell extends over the gum line of a user having an edentulous mouth.

5. The oral device of claim 2 wherein the plate-like bridge conforms to the back of the maxillary anteriors and the upper palate of the mouth, said bridge joining the biting surfaces.

6. The oral device of claim 1 further comprising:
means for releasably connecting the airway defined between the upper and lower members to a source of pressurized air, said connection means protruding from the device to a location external to the mouth of the user; and
at least one air direction member, the air direction member integrally formed in at least one of the upper or lower members and extending essentially perpendicular to the air channel defined by the upper and lower members.

7. The oral device of claim 6 wherein the at least one air direction foil is integrally connected to and protrudes from the upper surface of the lower member to a location proximate to the lower face of the upper member.

8. The device of claim 1 wherein the upper member comprises a plurality of tooth engaging regions, the tooth engaging regions including retentive portions extending over the super bulge of each of associated back molars and second bicuspids of the user and tooth-contacting regions positioned proximate to frontal regions of the device, the frontal tooth-contacting regions adapted to releasably contact at least a portion of inner surfaces of upper front teeth of the user.

9. The device of claim 8 wherein the lower member comprises a frontal shield extending angular to the lateral member to terminal positions above and below the lower member, the frontal shield having an interiorly oriented face adapted to releasably contact outer surfaces of associated front teeth, the frontal shield further having an opposed outwardly oriented surface, the opposed outwardly oriented surface having a upper region adapted to contact tissue associated with at least a portion of inner upper lip region of the user and a lower region, the lower region having a surface adapted to contact at least a portion of lower lip region of the user.

10. The device of claim 9 wherein the lower member further comprises a tooth engaging region integrally connected to the lower region of the frontal shield, the tooth engaging region releasably contacting at least one incisor located in the mandible of the user.

11. The device of claim 9 further comprising means for connecting the lower member to a source of pressurized air, the connecting means extending outward from the frontal shield member to a location external to the mouth of the user.

12. The device of claim 9 wherein the frontal shield further comprises a lateral protrusion adapted to releasably contact with a source of pressurized air and at least one orifice communicating with protrusion to a location in communication with the air channel formed by the upper and lower members.

13. The device of claim 1 further comprising means for connecting the upper member and the lower member in a releasably, essentially airtight manner.

14. The device of claim 13 wherein the releasable connecting means comprises at least one part of clip members extending outward from the anterior end of the lower member, the clip members deformably received in clip engaging channels defined in the anterior of the upper member.

15. An oral device removably insertable in the mouth of a user for facilitating breathing while sleeping. The device comprising:
an upper member, the upper member having upper and lower depressions and right and left sides corresponding to the location of at least back molars and second bicuspids, the upper member configured to fit a mouth of a user, the upper and lower depressions forming right and left biting surfaces wherein said biting surfaces are spaced at a height to force the mouth open to a position that protrudes the jaw, the upper member further having a plate-like bridge conforming to the back of the maxillary anterior and the upper palate of the mouth, the plate-like bridge contiguous connected between the biting surfaces;
a lower member positioned in underlying spaced relationship relative to the plate-like bridge of the upper member, the lower member having an upper surface oriented toward the plate-like bridge and a lower surface opposed to the upper surface, the lower member further having at least one curvilinear side surface extending between the upper and lower surfaces from a frontal location proximate the upper incisors to a terminal location proximate to the maxillary anterior of the user's mouth, the at least one side edge releasably connected to associated interiorly oriented regions of the upper member, the lower member positioned relative to the upper member so as to define an air passage for conveying pressurized air from the mouth opening to the anterior of the mouth of the user;
means for releasably connecting the airway defined between the upper and lower members to a source of pressurized air external to the oral device, the connection means extending from the device to a location external to the mouth where the device is in position.

16. The device of claim 15 wherein the air connection means is formed integral with the lower member and wherein the lower member further comprises a frontal shield extending angularly outward from the lateral member to terminal positions above and below the lower member, the frontal shield having an interiorly oriented face adapted to releasably contact outer surfaces of associated front teeth, the frontal shield further having an opposed outwardly oriented surface;
the opposed outwardly oriented surface having an upper region and a lower region;
the upper region adapted to contact tissue associated with at least a portion of inner upper lip region of the user;
the lower region having a surface adapted to contact at least a portion of associated lower incisors and an opposed surface adapted to contact at least a portion of lower lip region.

17. The device of claim 16 wherein the airway connection means comprises a channel member having an airway passage defined therein, the channel member communicating with the airway defined in the device at a location proximate to the frontal shield and releasably contacting with the source of pressurized air at a location distal to the frontal shield.

18. The device of claim 17 wherein the upper member further comprises an annular groove positioned in the interior face of the upper member at a location between the right and left sides and the plate-like bridge, the annular groove extending from the anterior surface of the upper member.

19. The device of claim 18 wherein the lower member comprises at least two clips extending from the anterior of the lower member and configured to be releasably contained in the annular groove defined in the upper member.

20. An oral device for facilitating breathing comprising:
a first member, the first member having a pair of tooth-engaging elements and a plate-like bridge positioned and contiguously formed with the tooth-engaging elements, wherein the tooth-engaging elements each have at least one depression defined in an upper face thereof, the depressions adapted to form biting surfaces and to removably receive at least upper back molars and upper bicuspids therein, the tooth-engaging elements further having a lower face opposed to the upper face;
a pair of tooth-engaging members having at least one depression defined therein, the depression adapted to form biting surfaces and to removably receive at least lower back molars and lower bicuspids therein, the tooth engaging members further having an upper face opposed to the tooth engaging depression;
means for adjustably connecting the first member and the tooth-engaging members, wherein the biting surfaces are spaced at a height to force the mouth open to a position which protrudes the jaw.

21. The oral device of claim 20 wherein the adjustable connecting means further comprises:
a connection plate having a body and at least one member protruding outward there from; and
at least one aperture defined in at least one of the first member and the tooth-engaging members to releasably receive the at least one protruding member and means for securing the connection plate in the at least one of the first member and the tooth-engaging members not receiving the protruding member.

22. The oral device of claim 21 further comprising:
a second member positioned in underlying relative to the plate-like bridge of the first member, the second member having a lateral member having an upper surface oriented toward the plate-like bridge, a lower surface opposed to the upper surface and at least one curvilinear side surface extending from a frontal location to a terminal location proximate to the maxiliary anterior of the oral cavity, the at least one side edge adapted to engage the first member to form an air passage for conveying pressurized air from an opening formed proximate to the mouth opening of the user to a position proximate to the anterior of the oral cavity, the at least one said edge adapted to engage the first member to form an air passage for conveying pressurized air from an opening formed proximate to the mouth opening of the user to a position proximate to the anterior of the mouth.

23. The oral device of claim 22 further comprising:
means for releasably connecting the air passage to a source of pressurized air. The connection means protruding from the device to a location external to the mouth of the user.
